(19) United States
(12) Patent Application Publication
(10) Pub. No.: US 2026/0268896 A1
(43) Pub. Date: Sep. 10, 2026

(54) SYSTEM

(71) Applicant: SOFTBANK GROUP CORP., Tokyo (JP)

(72) Inventor: Hiroshi KOYAMA, Tokyo (JP)

(73) Assignee: SOFTBANK GROUP CORP., Tokyo (JP)

(21) Appl. No.: 19/555,955

(22) Filed: Mar. 4, 2026

Related U.S. Application Data
(60) Provisional application No. 63/767,121, filed on Mar. 5, 2025.

(51) Int. Cl. G10L 15/18; A61B 5/16; A61B 5/31; G05B 13/02; G10L 15/22; G10L 15/30

(52) U.S. Cl. CPC G10L 15/1815; A61B 5/165; A61B 5/31; G05B 13/027; G10L 15/22; G10L 15/30; G10L 2015/223

(57) ABSTRACT

A system enabling users to acquire information and communicate hands-free is provided. A sensor unit, comprising extremely small equipment implanted in the head or worn externally on the face, detects brainwaves and voice to analyze the user's intent. An analysis unit interprets the user's intent using generative AI and acquires necessary information from the cloud. An output unit provides information auditorily and visually based on the analysis results. This allows users to efficiently perform tasks even while moving or when their hands are occupied. Furthermore, a communication unit manages data communication between terminals and servers, and a memory unit stores user profile information to provide personalized services. This significantly enhances the convenience and efficiency of users' lives.

### CROSS-REFERENCE TO RELATED APPLICATION

This application claims priority under 35 U.S.C. § 119 to U.S. Provisional Patent Application No. 63/767,121, filed on Mar. 5, 2025, the entire contents of which are incorporated herein by reference.

### BACKGROUND

### Technical Field

The present disclosure relates to a system.

### Related Art

Japanese Patent Application Publication Laid-Open (JP-A) No. 2022-180282 discloses a persona chatbot control method performed by at least one processor, comprising: a step of receiving a user utterance; a step of adding to the user utterance a prompt containing a description of the chatbot's persona and related instructions; a step of encoding the prompt; and a step of inputting the encoded prompt into a language model to generate a chatbot utterance in response to the user utterance.

A system and method for enabling users to efficiently and quickly perform tasks such as information acquisition and communication without using their hands, as these activities become increasingly important in modern society is disclosed. Conventional devices and interfaces require operation using hands or gaze, which is inconvenient, especially while moving or when both hands are occupied.

The system enables hands-free interaction with generative AI, acquisition of necessary information, and proposal of next actions by directly detecting electrical signals within the brain or voice using a chip implanted in the head or extremely small equipment attached externally to the face, and interpreting the user's intent. This allows users to obtain information, enjoy music, or communicate with others even while walking, significantly enhancing convenience and efficiency in daily life. Furthermore, by playing music and arranging background music based on user preferences, it aims to provide personalized experiences tailored to individual needs, thereby enhancing user satisfaction.

As a means to solve the problem, a system comprising: a sensor unit including extremely small equipment implanted in the head or attached externally to the face; an analysis unit including generative AI that receives signals from the sensor unit and analyzes the user's intent; and an output unit that acquires information based on instructions from the analysis unit and outputs it to the user via auditory and visual means is provided. In this system, the sensor unit includes multiple sensors for detecting electrical signals within the brain, self-talk, bone conduction, and sound waves, enabling real-time detection of the user's intentions or questions. The analysis unit interprets the user's intent using natural language processing technology and accesses databases or information sources in the cloud via a mobile network to obtain necessary information. The output unit provides information to the user audibly and visually based on instructions from the analysis unit. This allows the user to obtain information or receive suggestions for next actions hands-free. Thus, the user can efficiently and quickly obtain information and communicate even while moving or when both hands are occupied.

### BRIEF DESCRIPTION OF THE DRAWING

FIG. 1 is a conceptual diagram showing an example configuration of the data processing system according to the first embodiment.

FIG. 2 is a conceptual diagram showing an example of the main functional components of the data processing device and smart device according to the first embodiment.

FIG. 3 is a conceptual diagram showing an example configuration of a data processing system according to a second embodiment.

FIG. 4 is a conceptual diagram showing an example of the main functions of the data processing device and smart glasses according to the second embodiment.

FIG. 5 is a conceptual diagram showing an example configuration of a data processing system according to a third embodiment.

FIG. 6 is a conceptual diagram showing an example of the main functions of the data processing device and headset-type terminal according to the third embodiment.

FIG. 7 is a conceptual diagram showing an example configuration of a data processing system according to the fourth embodiment.

FIG. 8 is a conceptual diagram showing an example of the main functions of the data processing device and robot according to the fourth embodiment.

FIG. 9 shows an emotion map onto which multiple emotions are mapped.

FIG. 10 shows an emotion map onto which multiple emotions are mapped.

### DETAILED DESCRIPTION

The following describes an example embodiment of a system according to the present disclosure with reference to the accompanying drawings.

First, the terminology used in the following description is explained.

In the following embodiments, a processor (hereinafter simply referred to as a “processor”) may be a single arithmetic unit or a combination of multiple arithmetic units. Furthermore, the processor may be a single type of arithmetic unit or a combination of multiple types of arithmetic units. Examples of processing units include a CPU (Central Processing Unit), a GPU (Graphics Processing Unit), a GPGPU (General-Purpose Computing on Graphics Processing Units), an APU (Accelerated Processing Unit), and the like.

In the following embodiments, signed RAM (Random Access Memory) is a memory where information is temporarily stored and is used as working memory by the processor.

In the following embodiments, the signed storage is one or more non-volatile storage devices that store various programs and various parameters, etc. Examples of non-volatile storage devices include flash memory (SSD (Solid State Drive)), magnetic disks (e.g., hard disk), or magnetic tape.

In the following embodiments, the communication I/F (Interface) is an interface that includes a communication processor and an antenna, among other components. The communication I/F governs communication between multiple computers. Examples of communication standards applicable to the communication I/F include wireless communication standards such as 5G (5th Generation Mobile Communication System), Wi-Fi (registered trademark), or Bluetooth (registered trademark).

In the following embodiments, “A and/or B” is synonymous with “at least one of A and B.” That is, “A and/or B” may mean A alone, B alone, or a combination of A and B. Furthermore, in this specification, when three or more items are connected using “and/or,” the same concept applies as for “A and/or B”.

### First Embodiment

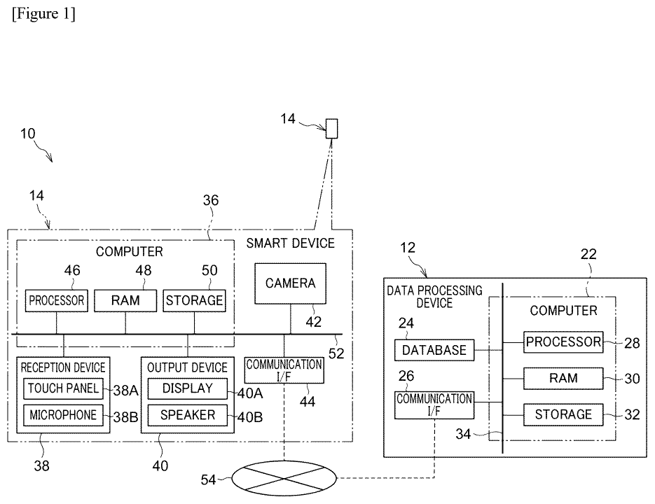

FIG. 1 shows an example configuration of a data processing system 10 according to the first embodiment.

As shown in FIG. 1, the data processing system 10 includes a data processing device 12 and a smart device 14. An example of the data processing device 12 is a server.

The data processing device 12 includes a computer 22, a database 24, and a communication I/F 26. The computer 22 is an example of a “computer” according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

The smart device 14 includes a computer 36, a reception device 38, an output device 40, a camera 42, and a communication I/F 44. The computer 36 includes a processor 46, RAM 48, and storage 50. The processor 46, RAM 48, and storage 50 are connected to a bus 52. The reception device 38, output device 40, and camera 42 are also connected to the bus 52. The reception device 38, output device 40, and camera 42 are also connected to the bus 52.

The reception device 38 includes a touch panel 38A and a microphone 38B, among other components, and receives user input. The touch panel 38A receives user input via contact with an indicator (e.g., a pen or finger) by detecting such contact. The microphone 38B receives voice-based user input by detecting the user's voice. The control unit 46A transmits data indicating the user input received via the touch panel 38A and microphone 38B to the data processing unit 12. Within the data processing unit 12, the specific processing unit 290 acquires the data indicating the user input.

Output device 40 includes display 40A and speaker 40B, presenting data to user 20 by outputting it in a perceptible form (e.g., audio and/or text). Display 40A displays visual information such as text and images according to instructions from processor 46. Speaker 40B outputs audio according to instructions from processor 46. Camera 42 is a compact digital camera equipped with an optical system including a lens, aperture, and shutter, and an imaging element such as a CMOS (Complementary Metal-Oxide-Semiconductor) image sensor or a CCD (Charge Coupled Device) image sensor.

The communication interface 44 is connected to the network 54. The communication interfaces 44 and 26 manage the exchange of various information between processor 46 and processor 28 via network 54.

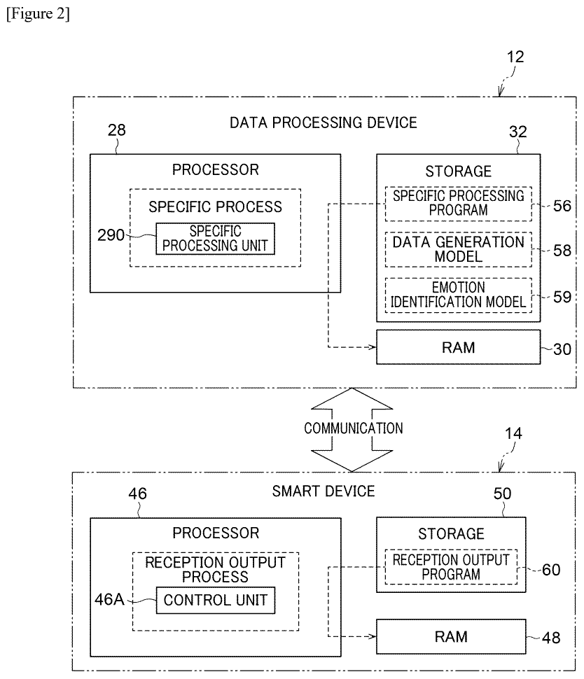

FIG. 2 shows an example of the main functions of the data processing device 12 and the smart device 14.

As shown in FIG. 2, specific processing is performed by processor 28 in data processing device 12. Specific processing program 56 is stored in storage 32. Specific processing program 56 is an example of a “program” pertaining to the technology of this disclosure. Processor 28 reads specific processing program 56 from storage 32 and executes the read specific processing program 56 in RAM 30. Program 56 is an example of a “program” related to the technology of this disclosure. Processor 28 reads specific processing program 56 from storage 32 and executes the read specific processing program 56 on RAM 30. The specific processing is realized by processor 28 operating as specific processing unit 290 according to specific processing program 56 executed on RAM 30.

Storage 32 stores a data generation model 58 and an emotion identification model 59. The data generation model 58 and emotion identification model 59 are used by specific processing unit 290. Specific processing unit 290 can estimate a user's emotion using emotion identification model 59 and perform specific processing using the user's emotion. The emotion estimation function (emotion identification function) using the emotion identification model 59 performs various estimations and predictions concerning the user's emotion, including estimation and prediction of the user's emotion, but is not limited to such examples. Furthermore, estimation and prediction of emotion also includes, for example, analysis (parsing) of emotion.

The smart device 14 performs reception output processing via the processor 46. The reception output program 60 is stored in the storage 50. The reception output program 60 is used in conjunction with the specific processing program 56 by the data processing system 10. The processor 46 reads the reception output program 60 from the storage 50 and executes the read reception output program 60 on the RAM 48. The specific processing is performed by the processor 46 operating as a control unit 46A according to the specific processing program 60 executed on the RAM 48. Note that the smart device 14 may also have data generation models and emotion identification models similar to the data generation model 58 and emotion identification model 59, and may perform processing similar to that of the specific processing unit 290 using these models. The reception output processing is realized by the processor 46 operating as the control unit 46A according to the reception output program 60 executed on the RAM 48.

Other devices besides the data processing device 12 may also have the data generation model 58. For example, a server device (e.g., a generation server) may have the data generation model 58. In this case, the data processing device 12 communicates with the server device having the data generation model 58 to obtain processing results (such as prediction results) obtained using the data generation model 58. Furthermore, the data processing device 12 may be the server device itself, or it may be a terminal device owned by a user (e.g., a mobile phone, robot, home appliance, etc.). Next, an example of processing by the data processing system 10 according to the first embodiment will be described.

### Example 1

The flow of the specific processing in Example 1 is described below. The components of the system described below are implemented by the data processing device 12 and the smart device 14. The data processing device 12 is referred to as the “server,” and the smart device 14 is referred to as the “terminal.”

### Implementation Examples for Carrying out the Invention

The embodiment for implementing the present invention will now be described in further detail. This system is realized through the cooperation of the server and the terminal, and details how each part functions.

First, the sensor unit is placed on the terminal side. This terminal includes extremely small equipment that is either implanted in the user's head or worn externally on the face. Specifically, it incorporates sensors such as an EEG sensor, a bone conduction sensor, and a sound wave sensor. The EEG sensor detects electrical signals generated within the user's brain in real time, providing data for analyzing the user's intentions and emotional state. For example, it can detect when the user is concentrating or relaxed and provide corresponding information or play music accordingly. The bone conduction sensor detects sound waves transmitted through the user's skull, providing data to analyze the user's mutterings or voice commands. This allows the user to convey clear instructions to the system without being affected by surrounding noise.

Next, the analysis unit is implemented on the server side. The server receives sensor data transmitted from the terminal and analyzes the user's intent using generative AI. This analysis employs advanced natural language processing technology to accurately interpret the user's thoughts or questions. For example, if the user thinks “What's my next appointment?” the server accesses a cloud-based calendar application, confirms the next scheduled event, and informs the user. Similarly, if the user thinks “Tell me about nearby cafes,” the server uses location services to search for the closest cafes, calculates the optimal route from the user's current location, and provides it. Furthermore, the server can learn from the user's past behavior and preferences to deliver personalized information.

Output is handled on the device side. Based on instructions from the server, the device provides information to the user through auditory and visual means. For example, if the user thinks, “Play relaxing music,” the device selects the optimal song from the user's music library or streaming services and plays it using bone conduction technology. This allows the user to enjoy music with clear sound quality while blocking out surrounding noise. Furthermore, if the user thinks “Send a message to my friend,” the terminal uses voice recognition technology to compose the message and sends it via the server. This enables the user to communicate quickly without using their hands.

Furthermore, this system can play music and arrange background music based on the user's preferences. For example, if the user thinks, “Make it a more energetic song,” the AI adjusts the music's tempo and rhythm to provide a musical experience matching the user's mood. Also, if the user thinks, “Add natural sounds to this song,” the AI can combine natural sounds like birdsong or the murmur of a river to generate new background music.

In this way, the system of the present invention enables users to obtain information and communicate without using their hands. Through the collaboration between the server and the terminal, users can efficiently and quickly perform tasks even while moving or when their hands are occupied. This significantly enhances the convenience and efficiency of users' lives and enables the provision of personalized experiences tailored to individual needs.

### System Configuration

The system according to this embodiment comprises a sensor unit, an analysis unit, an output unit, a communication unit, and a storage unit. The sensor unit includes extremely small equipment that is either implanted in the user's head or attached externally to the face, and is equipped with components such as an EEG sensor, a bone conduction sensor, and a sound wave sensor. The EEG sensor detects electrical signals generated within the user's brain in real time, providing data for analyzing the user's intentions and emotional state. For example, it can detect when the user is in a state of concentration or relaxation and provide corresponding information or play music accordingly. The bone conduction sensor detects sound waves transmitted through the user's skull and provides data for analyzing the user's mutterings or voice commands. This allows the user to convey clear instructions to the system without being affected by surrounding noise.

The analysis unit is implemented on the server side. It receives sensor data transmitted from the terminal and uses generative AI to analyze the user's intent. This analysis employs advanced natural language processing technology to accurately interpret the user's thoughts or questions. For example, if the user thinks, “What's my next appointment?”, the analysis unit accesses the calendar application in the cloud, confirms the next appointment, and informs the user. Similarly, if the user thinks “Tell me about nearby cafes,” the analysis unit uses location services to search for the nearest cafes, calculates the optimal route from the user's current location, and provides it. Furthermore, the analysis unit can learn from the user's past behavior history and preferences to provide personalized information. Specific examples of prompt sentences fed to the generative AI include: “The user wants to check their next schedule. Check the calendar and tell me the next appointment.” or “The user is looking for a nearby cafe. Search for the closest cafe from the current location and provide directions.”

The output unit is implemented on the device side and provides information to the user both audibly and visually based on instructions from the analysis unit. For example, if the user thinks “Play relaxing music,” the output unit selects the optimal song from the user's music library or streaming service and plays it using bone conduction technology. This allows the user to enjoy music with clear sound quality while blocking out surrounding noise. Furthermore, if the user thinks “Send a message to my friend,” the output unit uses voice recognition technology to compose the message and sends it via a server. This enables the user to communicate quickly without using their hands.

The communication unit manages data communication between the terminal and the server, sends data from the sensor unit to the analysis unit, and transmits the analysis results to the output unit. For example, if the user thinks “Download the materials for the next meeting,” the communication unit retrieves the materials from the server and transfers them to the terminal. Similarly, if the user thinks “Tell me the latest news,” the communication unit retrieves the news feed from the server and provides it to the user.

The memory unit stores user profile information, past activity history, preference settings, and other data, providing the foundation for the analysis unit and output unit to utilize this information to deliver personalized services. For example, when a user thinks, “Play my favorite playlist,” the memory unit retrieves the corresponding playlist from the user's music library and provides it to the output unit. Similarly, if a user thinks, “Show me the notes from the last meeting,” the memory unit searches the stored notes and displays them to the user.

In this way, the system according to this embodiment enables each component to collaborate to accurately interpret the user's intent and provide information quickly and efficiently. This allows the user to obtain information and communicate hands-free, significantly enhancing the convenience and efficiency of daily life.

### Implementation Steps

### Step 1: Collecting Sensor Data

In this step, the sensor unit embedded in the user's head detects the user's brain activity and voice commands using a brainwave sensor, a bone conduction sensor, and a sound wave sensor. The EEG sensor monitors the user's state of concentration or relaxation in real time, providing data for analyzing the user's intent. The bone conduction sensor detects sound waves transmitted through the user's skull, providing data for analyzing mutterings or voice commands. This allows the user to convey clear instructions to the system without being affected by surrounding noise.

### Step 2: Intent Analysis

In this step, the server-side analysis unit receives sensor data transmitted from the terminal and analyzes the user's intent using generative AI. Advanced natural language processing technology is employed to accurately interpret the user's thoughts or questions. For example, if the user thinks, “What's my next appointment?”, the analysis unit accesses the calendar application in the cloud, confirms the next appointment, and informs the user. Specific examples of prompt sentences fed to the generative AI include: “The user wants to check their next schedule. Check the calendar and tell them their next schedule.”

### Step 3: Information Acquisition and Provision

After the analysis unit interprets the user's intent, it acquires the necessary information and provides it to the user via the output unit. For example, if the user thinks, “Tell me about nearby cafes,” the analysis unit uses location services to search for the nearest cafes, calculates the optimal route from the user's current location, and provides it. If the user thinks, “Play relaxing music,” the output unit selects the optimal song from the user's music library or streaming services and plays the music using bone conduction technology.

### Step 4: Communication Management

The communication unit manages data communication between the terminal and the server. It transmits data from the sensor unit to the analysis unit and conveys the analysis results to the output unit. For example, if the user thinks, “Download the materials for my next meeting,” the communication unit retrieves the materials from the server and transfers them to the terminal. Similarly, if the user thinks, “Tell me the latest news,” the communication unit fetches the news feed from the server and provides it to the user.

### Step 5: Providing Personalized Services

The memory unit stores user profile information, past activity history, preference settings, and other data. It provides the foundation for the analysis unit and output unit to utilize this information to deliver personalized services. For example, if a user thinks, “Play my favorite playlist,” the memory unit retrieves the corresponding playlist from the user's music library and provides it to the output unit. Similarly, if the user thinks, “Show me the notes from the last meeting,” the storage unit searches the saved notes and displays them to the user.

### Specific Use Cases

For example, consider a user utilizing the present system during their morning commute. Brainwave sensors embedded in the user's head detect their brain activity and monitor their concentration state. When the user thinks, “I want to check today's schedule,” the sensor unit transmits this intent to the analysis unit. The analysis unit interprets the user's intent using generative AI, accesses the calendar application in the cloud, and retrieves the schedule for that day. A specific example of a prompt sentence fed to the generative AI is: “The user wants to check today's schedule. Please check the calendar and tell me about the schedule for today.”

Next, the analysis unit sends the retrieved schedule information to the output unit. The output unit provides the information to the user audibly using bone conduction technology. This allows the user to check the day's schedule hands-free. Furthermore, when the user thinks, “I want to check the materials for the next meeting,” the communication unit retrieves the materials from the server and transfers them to the terminal. The output unit presents the materials visually to the user, enabling the user to efficiently review the materials even while moving.

Furthermore, if the user thinks, “I want to listen to relaxing music,” the analysis unit accesses the user's music library and selects the optimal song based on past selection history and preferences. The output unit plays the selected music using bone conduction technology, allowing the user to enjoy the music without being bothered by surrounding noise. Specific examples of prompt sentences to feed into the generative AI include: “The user wants to listen to relaxing music. Please select the optimal song from the music library and play it.”

Thus, the system of the present invention enables users to acquire information hands-free in daily life and efficiently perform tasks. Users can quickly obtain necessary information even while moving, significantly enhancing convenience and efficiency in daily life.

### Application Example 1

The flow of specific processing in Application Example 1 is described below. The components of the system described below are implemented by the data processing device 12 and the smart device 14. The data processing device 12 is referred to as the “server,” and the smart device 14 is referred to as the “terminal.”

### Embodiments for Implementing the Present Invention

The embodiments for implementing the present invention will now be described in further detail. This system is designed to support the daily lives of users in the caregiving field and comprises a sensor unit, an analysis unit, an output unit, a communication unit, and a storage unit.

The sensor unit includes extremely small equipment that is either implanted in the user's head or attached externally to the face. Specifically, it incorporates an EEG sensor, a bone conduction sensor, and a sound wave sensor. The EEG sensor detects electrical signals generated within the user's brain in real time, monitoring changes in the user's health status and emotions. For example, if an increase in stress level is detected, the system can issue instructions to play relaxing music. Furthermore, when a relaxed state is detected, the environment can be adjusted to ensure the user's comfort. The bone conduction sensor detects sound waves transmitted through the user's skull, providing data to analyze muttered words or voice commands. This allows the user to give clear instructions to the system without being affected by surrounding noise. The acoustic sensor analyzes the tone and volume of the user's voice, enabling a more detailed understanding of the user's emotional state.

The analysis unit is implemented on the server side. It receives data transmitted from the sensor unit and uses generative AI to analyze the user's intent. This analysis employs advanced natural language processing technology to accurately interpret the user's thoughts and requests. For example, if the user thinks “I want to drink water,” the analysis unit instructs the care robot to provide water. Furthermore, if the user thinks “Tell me when it's time for my next medication,” the analysis unit checks the medication schedule and provides notification at the appropriate time. Additionally, the analysis unit learns the user's past behavioral history and preferences, enabling it to provide personalized information. For example, if the user thinks “What's on my schedule today?”, the analysis unit can refer to the calendar and announce the schedule via voice.

The output unit operates the care robot and home appliance control system based on instructions from the analysis unit. For example, if the user thinks, “Make the room warmer,” the output unit adjusts the heating system to maintain a comfortable room temperature. If the user thinks, “I want to listen to music,” the output unit activates the music playback system and plays music matching the user's preferences. This enables the user to live in a comfortable environment. Furthermore, the output unit can provide visual information. For example, if the user thinks “Show me the news,” the latest news on a display.

The communication unit manages data communication between the terminal and the server. It sends data from the sensor unit to the analysis unit and transmits the analysis results to the output unit. For example, if the user thinks “Download the materials for the next meeting,” the communication unit retrieves the materials from the server and transfers them to the terminal. Similarly, if the user thinks “Tell me the latest news,” the communication unit obtains the news feed from the server and provides it to the user. The communication unit employs encryption technology to transmit and receive data securely.

The storage unit saves user profile information, past activity history, preference settings, and other data, providing the foundation for the analysis unit and output unit to utilize this information and deliver personalized services. For example, if a user thinks, “Play my favorite playlist,” the storage unit retrieves the corresponding playlist from the user's music library and provides it to the output unit. Similarly, if a user thinks, “Show me the notes from the last meeting,” the storage unit searches the saved notes and displays them to the user. The memory unit strictly manages data access permissions to protect user privacy.

In this way, the system of the present invention enables each component to collaborate, accurately interpret the user's intent, and provide information quickly and efficiently. This allows users to obtain information and communicate hands-free, significantly enhancing the convenience and efficiency of daily life. Furthermore, it constantly monitors the user's health status and, if an abnormality is detected, automatically sends a notification to emergency contacts, enabling rapid response.

### System Configuration

The system according to this embodiment comprises a sensor unit, an analysis unit, an output unit, a communication unit, and a storage unit. The sensor unit includes extremely small equipment that is either implanted in the user's head or attached externally to the face. Specifically, it incorporates an EEG sensor, a bone conduction sensor, and a sound wave sensor. The EEG sensor detects electrical signals generated within the user's brain in real time, monitoring changes in the user's health status and emotions. For example, if it detects an increase in stress levels, the system can instruct the playback of relaxing music. Furthermore, when a relaxed state is detected, the environment can be adjusted to ensure the user's comfort. The bone conduction sensor detects sound waves transmitted through the user's skull, providing data to analyze muttered words or voice commands. This allows the user to give clear instructions to the system without being affected by surrounding noise. The acoustic sensor analyzes the tone and volume of the user's voice, enabling a more detailed understanding of the user's emotional state.

The analysis unit is implemented on the server side. It receives data transmitted from the sensor unit and uses generative AI to analyze the user's intent. This analysis employs advanced natural language processing technology to accurately interpret the user's thoughts and requests. For example, if the user thinks “I want to drink water,” the analysis unit instructs the care robot to provide water. Specific examples of prompt sentences fed into the generative AI include: “The user desires water. Please have the care robot provide water.” Furthermore, if the user thinks, “Tell me when it's time for my next medication,” the analysis unit checks the medication schedule and provides notification at the appropriate time. Additionally, the analysis unit can learn from the user's past behavioral history and preferences to provide personalized information. For example, if the user thinks, “What's on my schedule today?” the analysis unit can refer to the calendar and verbally inform the user of their schedule.

The output unit operates the care robot and home appliance control system based on instructions from the analysis unit. For example, if the user thinks, “Make the room warmer,” the output unit adjusts the heating system to maintain a comfortable room temperature. Similarly, if the user thinks, “I want to listen to music,” the output unit activates the music playback system and plays music matching the user's preferences. This enables the user to live in a comfortable environment. Furthermore, the output unit can also provide visual information. For instance, if the user thinks, “Show me the news,” it can display the latest news on a screen.

The communication unit manages data communication between the terminal and the server. It sends data from the sensor unit to the analysis unit and transmits the analysis results to the output unit. For example, if the user thinks “Download the materials for the next meeting,” the communication unit retrieves the materials from the server and transfers them to the terminal. Similarly, if the user thinks “Tell me the latest news,” the communication unit obtains the news feed from the server and provides it to the user. To ensure data security, the communication unit uses encryption technology for data transmission.

The storage unit saves user profile information, past activity history, preference settings, and other data, providing the foundation for the analysis unit and output unit to utilize this information and deliver personalized services. For example, if a user thinks, “Play my favorite playlist,” the storage unit retrieves the corresponding playlist from the user's music library and provides it to the output unit. Similarly, if a user thinks, “Show me the notes from the last meeting,” the storage unit searches the saved notes and displays them to the user. The memory unit strictly manages data access permissions to protect user privacy.

In this way, the system of the present invention enables each component to collaborate to accurately interpret the user's intent and provide information quickly and efficiently. This allows the user to obtain information and communicate hands-free, significantly enhancing the convenience and efficiency of daily life. Furthermore, it constantly monitors the user's health status and, if an abnormality is detected, automatically sends a notification to emergency contacts, enabling a rapid response.

### Implementation Steps

### Step 1: Sensor Data Collection

In this step, the sensor unit detects the user's brainwaves and voice in real time. The brainwave sensor monitors electrical signals generated within the user's brain to understand changes in health status and emotions. For example, if it detects an increase in stress levels, the system proceeds to the next step to encourage relaxation. The bone conduction sensor detects sound waves transmitted through the user's skull, providing data to analyze mutterings or voice commands uttered by the user. The acoustic sensor analyzes the tone and volume of the user's voice to gain a more detailed understanding of their emotional state.

### Step 2: Intent Analysis

The analysis unit receives data transmitted from the sensor unit and uses generative AI to analyze the user's intent. This analysis employs advanced natural language processing technology to accurately interpret the user's thoughts and requests. For example, if the user thinks “I want to drink water,” the analysis unit instructs the care robot to provide water. Specific examples of prompt sentences fed into the generative AI include: “The user desires water. Please have the care robot provide water.”

### Step 3: Information Provision and Environment Adjustment

The output unit operates the care robot and home appliance control system based on instructions from the analysis unit. For example, if the user thinks “Make the room warmer,” the output unit adjusts the heating system and maintain a comfortable room temperature. If the user thinks “I want to listen to music,” the output unit activates the music playback system and plays music matching the user's preferences. Furthermore, the output unit can also provide visual information. For example, if the user thinks “Show me the news,” it can display the latest news on a screen.

### Step 4: Data Communication Management

The communication unit manages data communication between the terminal and the server. It sends data from the sensor unit to the analysis unit and transmits the analysis results to the output unit. For example, if the user thinks “Download the materials for the next meeting,” the communication unit retrieves the materials from the server and transfers them to the terminal. Similarly, if the user thinks “Tell me the latest news,” the communication unit obtains the news feed from the server and provides it to the user. To ensure data security, the communication unit uses encryption technology for data transmission.

### Step 5: Providing Personalized Services

The memory unit stores user profile information, past activity history, preference settings, and other data, providing the foundation for the analysis unit and output unit to utilize this information and deliver personalized services. For example, when a user thinks, “Play my favorite playlist,” the memory unit retrieves the corresponding playlist from the user's music library and provides it to the output unit. Similarly, when a user thinks, “Show me the notes from the last meeting,” the memory unit searches the stored notes and displays them to the user. The memory unit strictly manages data access permissions to protect user privacy.

### Specific Use Cases

For example, consider a user utilizing this system during daily life at home. Upon waking in the morning, the sensor unit monitors the user's sleep state via a brainwave sensor and activates the alarm at an appropriate time. When the user thinks “I want to know the morning news,” the analysis unit interprets this intent and instructs the communication unit to retrieve the latest news. A specific example of a prompt to feed to the generative AI is: “The user wants to know the morning news. Retrieve the latest news and display it on the output unit.”

The analysis unit retrieves news feeds from the server and visually displays them on the screen via the output unit. If the user thinks “I want to check today's schedule,” the analysis unit accesses the calendar application and retrieves the day's schedule. This allows the user to check their schedule hands-free.

Furthermore, if the user thinks “I want breakfast prepared,” the analysis unit instructs the care robot to prepare breakfast according to the user's preferences. A specific example of a prompt to feed into the generative AI is: “The user desires breakfast. Have the care robot prepare breakfast.” The care robot references the user's dietary preferences and allergy information stored in the memory unit to select an appropriate menu.

Furthermore, if the user thinks, “I want to listen to relaxing music,” the analysis unit accesses the music library and selects music matching the user's preferences. The output unit plays the selected music using bone conduction technology, allowing the user to enjoy the music without being bothered by surrounding noise. A specific example of a prompt to feed into the generative AI could be: “The user wants to listen to relaxing music. Please select the optimal song from the music library and play it.”

Thus, the system of the present invention enables users to acquire information hands-free in daily life and efficiently perform tasks. Users can quickly obtain necessary information even while moving, significantly enhancing convenience and efficiency in daily life. Furthermore, by constantly monitoring the user's health status, it enables prompt response by automatically sending notifications to emergency contacts when abnormalities are detected.

The specific processing unit 290 transmits the results of the specific processing to the smart device 14. In the smart device 14, the control unit 46A instructs the output device 40 to output the results of the specific processing. The microphone 38B acquires audio indicating user input regarding the results of the specific processing. The control unit 46A transmits the audio data indicating the user input acquired by the microphone 38B to the data processing unit 12. At the data processing unit 12, the specific processing unit 290 acquires the audio data.

The data generation model 58 is a so-called generative AI (Artificial Intelligence). An example of the data generation model 58 is ChatGPT (registered trademark) (Internet search <URL: https://openai.com/blog/chatgpt>). The data generation model 58 is obtained by performing deep learning on a neural network. The data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned components is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit may acquire step count data using the camera 42 or communication I/F 44 of the smart device 14, and this data is processed by the specific processing unit 290 of the data processing device 12. For example, the analysis unit may analyze the acquired step count data. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using the generated AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the smart device 14.

### Second Embodiment

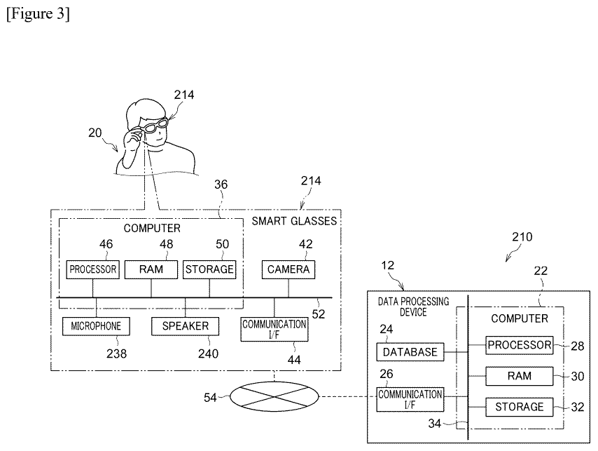

FIG. 3 shows an example configuration of the data processing system 210 according to the second embodiment.

As shown in FIG. 3, the data processing system 210 includes a data processing device 12 and smart glasses 214. An example of the data processing device 12 is a server.

The data processing device 12 includes a computer 22, a database 24, and a communication I/F 26. The computer 22 is an example of a “computer” according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

The smart glasses 214 include a computer 36, a microphone 238, a speaker 240, a camera 42, and a communication I/F 44. The computer 36 includes a processor 46, RAM 48, and storage 50. Processor 46, RAM 48, and storage 50 are connected to bus 52. Microphone 238, speaker 240, and camera 42 are also connected to bus 52.

Microphone 238 receives voice input from user 20 to accept instructions or other commands. Microphone 238 captures the voice input from user 20, converts the captured voice into audio data, and outputs it to processor 46. Speaker 240 outputs audio in accordance with instructions from processor 46.

Camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures images of the user's surroundings (e.g., an imaging range defined by a field of view equivalent to that of a typical healthy person).

The communication I/F 44 is connected to the network 54. The communication I/Fs 44 and 26 handle the exchange of various information between processor 46 and processor 28 via network 54. The exchange of various information between processor 46 and processor 28 using communication I/F 44 and 26 is performed in a secure state.

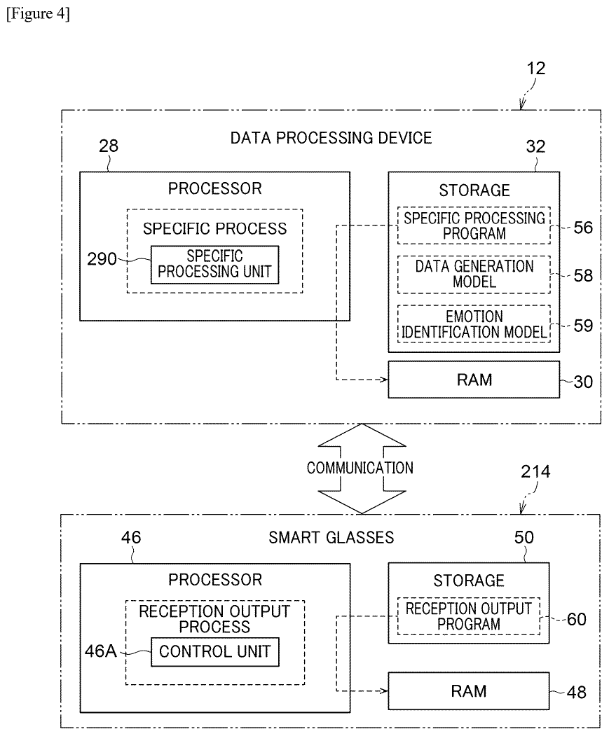

FIG. 4 shows an example of the main functions of the data processing device 12 and the smart glasses 214. As shown in FIG. 4, specific processing is performed by the processor 28 in the data processing device 12. The specific processing program 56 is stored in the storage 32.

The specific processing program 56 is an example of a “program” related to the technology of this disclosure. Processor 28 reads the specific processing program 56 from storage 32 and executes the read specific processing program 56 on RAM 30. The specific processing is realized by processor 28 operating as specific processing unit 290 according to the specific processing program 56 executed on RAM 30.

Storage 32 stores a data generation model 58 and an emotion identification model 59. The data generation model 58 and emotion identification model 59 are used by specific processing unit 290. Specific processing unit 290 can estimate a user's emotion using emotion identification model 59 and perform specific processing using the user's emotion. The emotion estimation function (emotion identification function) using the emotion identification model 59 performs various estimations and predictions concerning the user's emotion, including estimation and prediction of the user's emotion, but is not limited to such examples. Furthermore, estimation and prediction of emotion also includes, for example, analysis (parsing) of emotion.

The smart glasses 214 perform reception output processing via the processor 46. The reception output program 60 is stored in the storage 50. The processor 46 reads the reception output program 60 from the storage 50 and executes the read reception output program 60. Reception output processing is realized by the processor 46 operating as control unit 46A according to reception output program 60 executed on RAM 48. Furthermore, the smart glasses 214 may also have a data generation model 58 and an emotion identification model 59, and can perform processing similar to that of the identification processing unit 290 using these models.

Next, the identification processing performed by the identification processing unit 290 of the data processing device 12 is described. The components of the system described below are implemented by the data processing device 12 and the smart glasses 214. In the following description, the data processing device 12 is referred to as the “server,” and the smart glasses 214 are referred to as the “terminal.”

### Example 1

The flow of the specific processing is the same as that described in Example 1 of the first embodiment, so the description is omitted.

### Application Example 1

The flow of the specific processing in Example 1 described in the first embodiment is the same as above, so the description is omitted.

The specific processing unit 290 transmits the result of the specific processing to the smart glasses 214. In the smart glasses 214, the control unit 46A causes the speaker 240 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

The data generation model 58 is a so-called generative AI (Artificial Intelligence). An example of the data generation model 58 is ChatGPT (registered trademark) (Internet search <URL: https://openai.com/blog/chatgpt>). The data generation model 58 is obtained by performing deep learning on a neural network. The data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58. The data generation model 58 includes AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned components is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the acquisition unit is implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the smart glasses 214.

### Third Embodiment

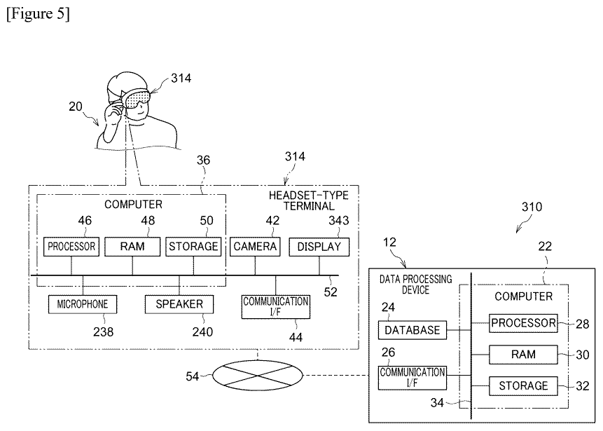

FIG. 5 shows an example configuration of the data processing system 310 according to the third embodiment.

As shown in FIG. 5, the data processing system 310 includes a data processing device 12 and a headset-type terminal 314. An example of the data processing device 12 is a server.

The data processing device 12 includes a computer 22, a database 24, and a communication I/F 26. The computer 22 is an example of a “computer” according to the technology of the present disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

The headset-type terminal 314 includes a computer 36, a microphone 238, a speaker 240, a camera 42, a communication interface 44, and a display 343. The computer 36 includes a processor 46, RAM 48, and storage 50. The processor 46, RAM 48, and storage 50 are connected to a bus 52. The microphone 238, speaker 240, camera 42, and display 343 are also connected to the bus 52.

Microphone 238 receives voice input from user 20 to accept instructions or other commands. Microphone 238 captures the voice input from user 20, converts the captured voice into audio data, and outputs it to processor 46. Speaker 240 outputs audio in accordance with instructions from processor 46.

The camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures images of the user's surroundings (e.g., an imaging range defined by a field of view equivalent to that of a typical healthy person).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

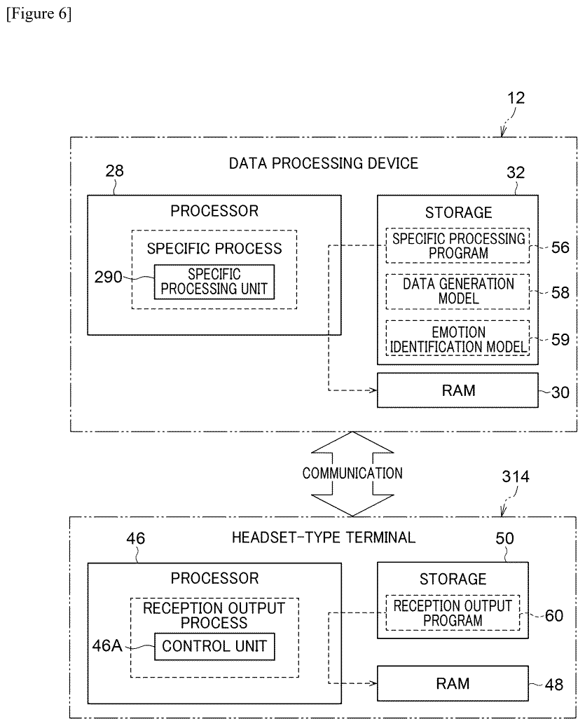

FIG. 6 shows an example of the main functions of the data processing device 12 and the headset-type terminal 314. As shown in FIG. 6, specific processing is performed by the processor 28 in the data processing device 12. The specific processing program 56 is stored in the storage 32.

The specific processing program 56 is an example of a “program” related to the technology of this disclosure. The processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and emotion identification model 59 are used by the specific processing unit 290.

In the headset terminal 314, reception output processing is performed by the processor 46. The reception output program 60 is stored in the storage 50. Processor 46 reads the reception output program 60 from storage 50 and executes the read reception output program 60 on RAM 48. Reception output processing is achieved by processor 46 operating as control unit 46A according to the reception output program 60 executed on RAM 48.

Next, the specific processing performed by the specific processing unit 290 of the data processing device 12 is described. The various parts of the system described below are implemented by the data processing device 12 and the headset-type terminal 314. In the following description, the data processing device 12 is referred to as the “server,” and the headset-type terminal 314 is referred to as the “terminal.”

### Example 1

The flow of the specific processing in Example 1 described in the above first embodiment is the same, so the explanation is omitted.

### Application Example 1

The flow of the specific processing in Example 1 described in the above first embodiment is the same, so the explanation is omitted.

The specific processing unit 290 transmits the result of the specific processing to the headset-type terminal 314. At the headset-type terminal 314, the control unit 46A causes the speaker 240 and the display 343 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

The data generation model 58 is a so-called generative AI (Artificial Intelligence). An example of the data generation model 58 is ChatGPT (registered trademark) (Internet search <URL: https://openai.com/blog/chatgpt>). The data generation model 58 is obtained by performing deep learning on a neural network. The data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data generation model 58 includes multiple types, and the data generation model 58 includes AI other than generation AI. AI other than generation AI includes, for example, linear regression, logistic regression, decision trees, and others. The data processing device 12 may include multiple types of data generation models 58, and the data generation model 58 may include AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned parts is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing performed by the data processing system 10 described above is executed by either the specific processing unit 290 of the data processing device 12 or the control unit 46A of the smart device 14, but it may also be executed by both the specific processing unit 290 of the data processing device 12 and the control unit 46A of the smart device 14. Furthermore, the specific processing unit 290 of the data processing device 12 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the headset-type terminal 314.

### Fourth Embodiment

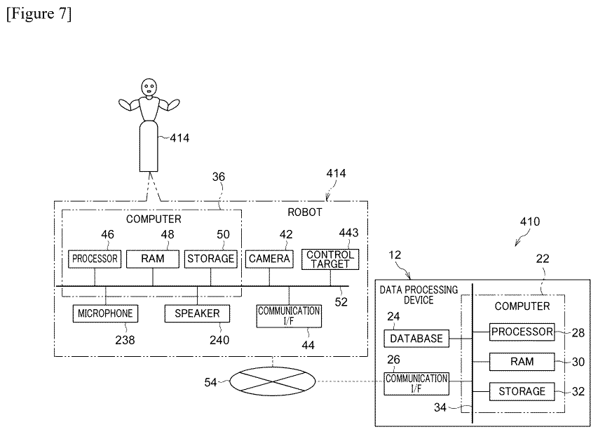

FIG. 7 shows an example configuration of the data processing system 410 according to the fourth embodiment.

As shown in FIG. 7, the data processing system 410 includes a data processing device 12 and a robot. It is equipped with 414. An example of the data processing device 12 is a server.

The data processing device 12 includes a computer 22, a database 24, and a communication I/F 26. The computer 22 is an example of a “computer” related to the technology of this disclosure. The computer 22 includes a processor 28, RAM 30, and storage 32. The processor 28, RAM 30, and storage 32 are connected to a bus 34. The database 24 and communication I/F 26 are also connected to the bus 34. The communication I/F 26 is connected to a network 54. An example of the network 54 include a WAN (Wide Area Network) and/or a LAN (Local Area Network).

Robot 414 includes a computer 36, a microphone 238, a speaker 240, a camera 42, a communication I/F 44, and a control target 443. Computer 36 includes a processor 46, RAM 48, and storage 50. Processor 46, RAM 48, and storage 50 are connected to bus 52. Furthermore, microphone 238, speaker 240, camera 42, and controlled object 443 are also connected to bus 52.

Microphone 238 receives voice commands from user 20 by capturing the user's spoken words. Microphone 238 captures the user's voice, converts the captured voice into audio data, and outputs it to processor 46. Speaker 240 outputs audio according to instructions from processor 46.

Camera 42 is a compact digital camera equipped with an optical system, such as a lens, aperture, and shutter, and an imaging element, such as a CMOS (Complementary Metal-Oxide-Semiconductor) image sensor or a CCD (Charge Coupled Device) image sensor. It captures images of the user's surroundings (e.g., an imaging range defined by a field of view equivalent to that of a typical healthy person).

The communication I/F 44 is connected to the network 54. The communication I/Fs 44 and 26 manage the exchange of various information between processor 46 and processor 28 via network 54. The exchange of various information between processor 46 and processor 28 using communication I/F 44 and 26 is performed in a secure state.

The control target 443 includes a display device, LEDs for the eye section, and motors for driving the arms, hands, legs, etc. The posture and gestures of robot 414 are controlled by controlling the motors for the arms, hands, legs, etc. Part of the robot 414's emotions can be expressed by controlling these motors. Furthermore, the robot 414's facial expressions can also be expressed by controlling the light emission state of the LEDs in its eyes.

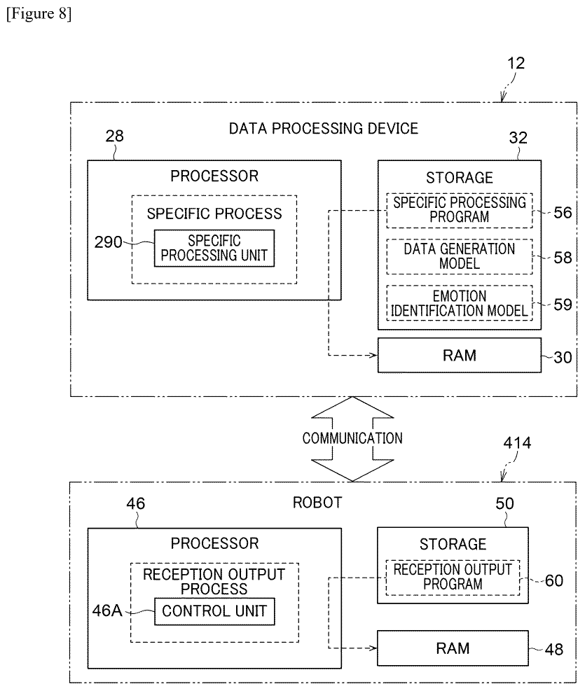

FIG. 8 shows an example of the main functions of the data processing device 12 and the robot 414. As shown in FIG. 8, specific processing is performed by the processor 28 in the data processing device 12. The specific processing program 56 is stored in the storage 32.

The specific processing program 56 is an example of a “program” pertaining to the technology of this disclosure. The processor 28 reads the specific processing program 56 from the storage 32 and executes the read specific processing program 56 on the RAM 30. The specific processing is realized by the processor 28 operating as a specific processing unit 290 according to the specific processing program 56 executed on the RAM 30.

Storage 32 stores a data generation model 58 and an emotion identification model 59. The data generation model 58 and the emotion identification model 59 are used by the specific processing unit 290.

In robot 414, reception output processing is performed by processor 46. Storage 50 stores a reception output program 60. Processor 46 reads the reception output program 60 from storage 50 and executes the read reception output program 60 on RAM 48. Reception output processing is realized by the processor 46 operating as a control unit 46A according to the reception output program 60 executed on RAM 48.

Next, the specific processing performed by the specific processing unit 290 of the data processing device 12 is described. The various parts of the system described below are implemented by the data processing device 12 and the robot 414. In the following description, the data processing device 12 is referred to as the “server,” and the robot 414 is referred to as the “terminal.”

### Example 1

The flow of the specific processing is the same as that described in Example 1 of the first embodiment, so the description is omitted.

### Application Example 1

The flow of the specific processing in Example 1 described in the above first embodiment is the same, so the explanation is omitted.

The specific processing unit 290 transmits the result of the specific processing to the robot 414. In the robot 414, the control unit 46A causes the speaker 240 and the control target 443 to output the result of the specific processing. The microphone 238 acquires audio indicating user input regarding the result of the specific processing. The control unit 46A transmits the audio data indicating the user input acquired by the microphone 238 to the data processing device 12. At the data processing device 12, the specific processing unit 290 acquires the audio data.

The data generation model 58 is a so-called generative AI (Artificial Intelligence). An example of the data generation model 58 is ChatGPT (registered trademark) (Internet search <URL: https://openai.com/blog/chatgpt>). The data generation model 58 is obtained by performing deep learning on a neural network. The data generation model 58 receives input of a prompt containing instructions, as well as inference data such as audio data representing sound, text data representing text, and image data (e.g., still image data or video data) representing images. The data generation model 58 infers based on the input inference data according to the instructions indicated by the prompt and outputs the inference result in one or more data formats, such as audio data, text data, and image data. The data generation model 58 includes, for example, text generation AI, image generation AI, and multimodal generation AI. Here, inference refers to, for example, analysis, classification, prediction, and/or summarization. The specific processing unit 290 performs the aforementioned specific processing while utilizing the data generation model 58. The data generation model 58 may be a fine-tuned model capable of outputting inference results from prompts that do not contain instructions. In this case, the data generation model 58 can output inference results from prompts that do not contain instructions. The data processing device 12 and the like may include multiple types of data generation models 58. The data generation model 58 includes AI other than generative AI. AI other than generative AI includes, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others. The AI may perform various processing operations, but is not limited to such examples. Furthermore, the AI may be an AI agent. Also, when the processing of the aforementioned components is performed by AI, that processing may be performed in part or in whole by AI, but is not limited to such examples. Furthermore, processing performed by AI, including generative AI, may be replaced by rule-based processing, and rule-based processing may be replaced by processing performed by AI, including generative AI.

Furthermore, the processing by the data processing system 10 described above may be executed by the specific processing unit 290 of the data processing device 12 or by the control unit 46A of the smart device 14. Alternatively, the processing may be executed by the specific processing unit 290 of the data processing device 12 and the control unit 46A of the smart device 14. Furthermore, the data processing device 12's specific processing unit 290 acquires or collects information necessary for processing from the smart device 14 or external devices, etc., and the smart device 14 acquires or collects information necessary for processing from the data processing device 12 or external devices, etc.

For example, the collection unit may be implemented by the control unit 46A of the smart device 14 or the specific processing unit 290 of the data processing device 12. For example, the acquisition unit acquires step count data using the camera 42 or communication I/F 44 of the smart device 14, and this data is processed by the specific processing unit 290 of the data processing device 12. For example, the analysis unit is implemented by the specific processing unit 290 of the data processing device 12 and analyzes data from the collection unit and acquisition unit. For example, the generation unit is implemented by the specific processing unit 290 of the data processing device 12 and generates a cooking menu using a generation AI. For example, the provision unit is implemented by the output device 40 of the smart device 14 or the specific processing unit 290 of the data processing device 12 and provides the generated cooking menu to the user. The correspondence between each unit and the device or control unit is not limited to the above examples and various modifications are possible.

The above embodiment described a form where specific processing is performed by the data processing device 12, but the technology disclosed herein is not limited thereto; specific processing may also be performed by the robot 414.

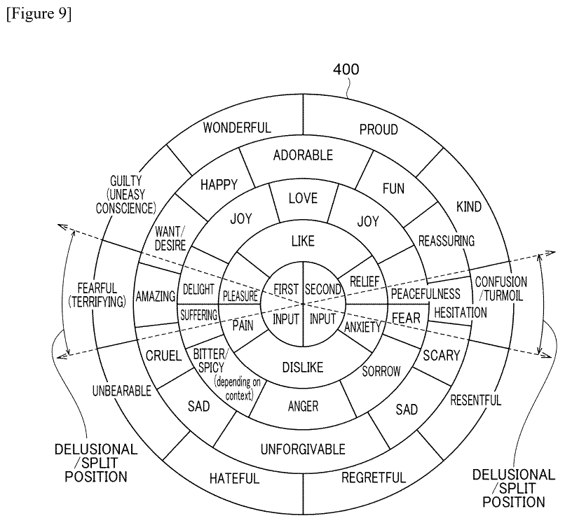

The emotion identification model 59, functioning as an emotion engine, may determine the user's emotion according to a specific mapping. Specifically, the emotion identification model 59 may determine the user's emotion according to an emotion map (see FIG. 9), which is a specific mapping. Furthermore, the emotion identification model 59 may similarly determine the robot's emotion, and the specific processing unit 290 may perform specific processing using the robot's emotion.

FIG. 9 is a diagram showing an emotion map 400 where multiple emotions are mapped. In the emotion map 400, emotions are arranged radially in concentric circles from the center. Emotions closer to the center of the concentric circles represent more primitive states. Emotions representing states or behaviors arising from mental states are placed further out in the concentric circles. Emotion is a concept encompassing affect and mental states. Generally, emotions generated from reactions occurring within the brain are placed on the left side of the concentric circles. Generally, emotions induced by situational judgment are placed on the right side of the concentric circles. Generally, emotions generated from reactions occurring within the brain and also induced by situational judgment are placed on the upper and lower sides of the concentric circles. Furthermore, the upper part of the concentric circle contains “pleasant” emotions, while the lower part contains “unpleasant” emotions. Thus, the Emotion Map 400 maps multiple emotions based on the structure of their origin, with emotions that tend to occur simultaneously mapped close together.

These emotions are distributed around the 3 o'clock position on Emotion Map 400, typically oscillating between feelings of security and anxiety. In the right half of Emotion Map 400, situational awareness takes precedence over internal sensations, resulting in a calmer impression.

The inner part of the emotion map 400 represents the mind, while the outer part represents behavior. Therefore, the further outward one goes on the emotion map 400, the more visible the emotion becomes (manifesting in behavior).

Here, human emotions are based on various balances, such as posture and blood sugar levels. When these balances deviate from the ideal, it results in discomfort; when they approach the ideal, it results in pleasure. Similarly, in robots, automobiles, motorcycles, and other systems, emotions can be created based on various balances, such as posture and battery level. When these balances deviate from the ideal, it results in discomfort; when they approach the ideal, it results in pleasure. Emotion maps, such as Dr. Mitsuyoshi's Emotion Map (Research on Speech Emotion Recognition and Neurophysiological Signal Analysis of Emotions, Tokushima University, Doctoral Dissertation: https://ci.nii.ac.jp/naid/500000375379). The left half of the emotion map displays emotions belonging to the “Reaction” domain, where sensory perception dominates. The right half of the emotion map displays emotions belonging to the “Situation” domain, where situational awareness is dominant.

Two emotions that promote learning are defined in the emotion map. One is the negative emotion around the center of the “repentance” or “reflection” area on the situation side. That is, when the robot experiences negative emotions like “I never want to feel this way again” or “I don't want to be scolded anymore. ”The other is the positive emotion around “desire” on the reaction side. That is, when the robot feels positive emotions like “I want more” or “I want to know more.”

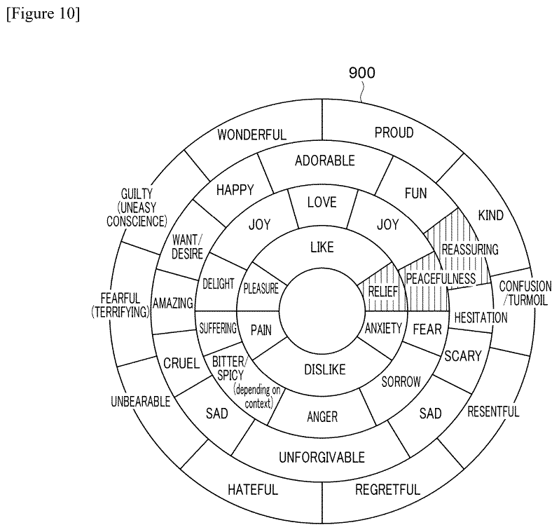

The emotion identification model 59 inputs the user input into a pre-trained neural network, obtains emotion values corresponding to each emotion shown in the emotion map 400, and determines the user's emotion. This neural network is pre-trained based on multiple training data sets, which are combinations of user input and emotion values corresponding to each emotion shown in the emotion map 400. Furthermore, this neural network is trained such that emotions positioned close to each other, as shown in Emotion Map 900 in FIG. 10, have similar values. FIG. 10 illustrates an example where multiple emotions, such as “reassurance,” “tranquility,” and “encouragement,” have similar emotion values.

The above description primarily explains the system of the present disclosure in terms of the functions of the data processing device 12. However, the system of the present disclosure is not necessarily implemented on a server. The system of the present disclosure may be implemented as a general information processing system. For example, the present disclosure may be implemented as a software program operating on a personal computer or as an application operating on a smartphone, etc. The method of the present disclosure may be provided to users in a SaaS (Software as a Service) format.

The above embodiments illustrated a configuration where specific processing is performed by a single computer 22. However, the technology of this disclosure is not limited thereto. Distributed processing may be performed by multiple computers, including computer 22, for specific processing. For example, data generation model 58 may be provided in an external device of data processing device 12, and said external device may generate data corresponding to input data. For example, the data generation model 58 may be provided in an external device of the data processing device 12, and data generation corresponding to input data may be performed in said external device.

The above embodiment described a configuration where a specific processing program 56 is stored in storage 32, but the technology disclosed herein is not limited thereto. For example, the specific processing program 56 may be stored on a portable, computer-readable non-volatile storage medium such as a USB (Universal Serial Bus) memory. The specific processing program 56 stored on the non-volatile storage medium is installed on the computer 22 of the data processing device 12. The processor 28 executes specific processing according to the specific processing program 56.

Alternatively, the specific processing program 56 may be stored on a storage device, such as a server, connected to the data processing device 12 via the network 54. Upon request from the data processing device 12, the specific processing program 56 is downloaded and installed on the computer 22.

It should be noted that it is not necessary to store the entire specific processing program 56 in a storage device such as a server connected to the data processing device 12 via the network 54, or to store the entire specific processing program 56 in the storage 32. It is also possible to store only a portion of the specific processing program 56.

Various types of processors can be used as hardware resources to execute the specific processing. Examples of processors include a CPU, which is a general-purpose processor that functions as a hardware resource for executing specific processing by executing software, i.e., a program. Additionally, processors may include dedicated electronic circuits, such as FPGAs (Field-Programmable Gate Array), PLDs (Programmable Logic Device), or ASICs (Application Specific Integrated Circuit), which are processors with circuit configurations specifically designed to execute particular processing tasks. Each processor incorporates or connects to memory, and each processor executes specific processing by utilizing this memory.

The hardware resources for executing specific processing may be comprised of one of these various processors, or may be comprised of a combination of two or more processors of the same or different types (e.g., a combination of multiple FPGAs, or a combination of a CPU and an FPGA). Furthermore, the hardware resources for executing specific processing may be a single processor.

Examples of configurations using a single processor include: First, a configuration where one processor is formed by combining one or more CPUs with software, with this processor functioning as the hardware resource executing specific processing. Second, there is a form using a processor that implements the entire system's functionality, including multiple hardware resources executing specific processing, on a single IC chip, as exemplified by a System-on-a-chip (SoC). Thus, specific processing is implemented as a hardware resource using one or more of the various processors described above.

Furthermore, regarding the hardware structure of these various processors, more specifically, electrical circuits combining circuit elements such as semiconductor devices can be used. Also, the specific processing described above is merely one example. Therefore, it goes without saying that within the scope not deviating from the main purpose, unnecessary steps may be omitted, new steps may be added, or the processing order may be changed.

The above description and illustrations provide a detailed explanation of the aspects pertaining to the technology of the present disclosure and represent merely one example of the technology disclosed herein. For example, the above descriptions of the configuration, functions, actions, and effects are merely examples of the configuration, functions, actions, and effects pertaining to the technology disclosed herein. Therefore, it goes without saying that within the scope that does not deviate from the spirit of the technology disclosed herein, unnecessary portions may be omitted, new elements may be added, or replacements may be made to the above-described content and illustrated content. Furthermore, to avoid complexity and facilitate understanding of the technical aspects of the present disclosure, descriptions of common technical knowledge and the like that are not particularly necessary for enabling the present disclosure to have been omitted from the above descriptions and illustrations.

All literature, patent applications, and technical specifications cited herein are incorporated by reference to the same extent as if each were specifically and individually cited herein.

The following is further disclosed regarding the above embodiments.

### Supplementary Note 1

A system comprising a sensor unit, an analysis unit, and an output unit. The sensor unit includes extremely small equipment that is either implanted in the user's head or attached externally to the face, and is equipped with an EEG sensor, a bone conduction sensor, and a sound wave sensor, enabling real-time detection of the user's brain activity and voice commands. The analysis unit operates on the server side, receives data transmitted from the sensor unit, analyzes the user's intent using generative AI, and determines necessary actions. The output unit operates care robots or home appliance control systems based on instructions from the analysis unit, providing information to the user through auditory and visual means.

The sensor unit monitors the user's health status and emotional changes, detecting increases in stress levels or states of relaxation. The voice sensor recognizes the user's mutterings and voice commands, providing data to understand the user's needs.

The analysis unit interprets the user's intent using generative AI, accesses a database in the cloud to obtain necessary information, and the output unit operates care robots or home appliance control systems based on the analysis results to provide personalized services to the user, as described in Supplementary Note 1.

### Explanation of Symbols

10, 210, 310, 410 Data Processing System
12 Data Processing Device
14 Smart Device
214 Smart Glasses
314 Headset-Type Devices
414 Robot

What is claimed is

1. A hands-free information processing system, comprising:
a sensor unit disposed in extremely small equipment that is either implanted in a user's head or attached externally to a face of the user, the sensor unit including:
an EEG sensor configured to detect electrical signals generated within a brain of the user in real time,
a bone conduction sensor configured to detect sound waves transmitted through a skull of the user, and
a sound wave sensor configured to detect voice input of the user;
a communication unit configured to transmit data detected by the sensor unit to a server via a network;
an analysis unit implemented on the server and configured to:
receive the transmitted data, input the received data into a generative AI model,
analyze an intent of the user using natural language processing, and
determine an action corresponding to the analyzed intent; and
an output unit configured to operate at least one of a care robot or a home appliance control system based on the determined action and to provide information to the user audibly or visually.

2. The system of claim 1, wherein the EEG sensor monitors a concentration state or stress level of the user and provides corresponding data to the analysis unit.

3. The system of claim 1, wherein the analysis unit accesses a cloud-based calendar application, news source, or location service based on the analyzed intent.

4. The system of claim 1, further comprising a storage unit configured to store user profile information, past activity history, and preference settings, and wherein the analysis unit utilizes the stored information to determine the action.

5. The system of claim 1, wherein the generative AI model receives a prompt including instructions corresponding to the analyzed intent.

6. The system of claim 1, wherein the communication unit transmits data using encryption technology.

7. A computer-implemented method for hands-free interaction, comprising:
detecting, by a sensor unit including extremely small equipment implanted in a user's head or attached externally to a face, brainwave signals using an EEG sensor and voice-related signals using a bone conduction sensor and a sound wave sensor;
transmitting the detected signals to a server;
analyzing, by an analysis unit implemented on the server, the transmitted signals using a generative AI model to determine a user intent;
determining, based on the determined user intent, at least one of:
retrieving information from an external information source, or
generating control instructions for a care robot or a home appliance control system; and
outputting information to the user audibly or visually without requiring manual input.

8. The method of claim 7, further comprising estimating an emotional state of the user using an emotion identification model trained based on mapped emotion values.

9. The method of claim 7, wherein retrieving information includes accessing a cloud-based database.

10. The method of claim 7, wherein generating control instructions includes adjusting an environmental condition or initiating operation of a robot.

11. The method of claim 7, wherein the generative AI model processes at least one of audio data, text data, or image data.

12. The method of claim 7, further comprising storing user behavioral history and utilizing the stored history to personalize subsequent actions.

13. A non-transitory computer-readable storage medium storing instructions that, when executed by at least one processor, cause the processor to:
receive sensor data representing brainwave signals detected by an EEG sensor and voice-related signals detected by a bone conduction sensor and a sound wave sensor, the sensors being included in extremely small equipment implanted in a user's head or attached externally to a face;
transmit the received sensor data to a server;
analyze the sensor data using a generative AI model to determine a user intent;
determine an action corresponding to the user intent, including retrieving information or operating a care robot or home appliance control system; and
cause output of information to the user via auditory or visual means.

14. The storage medium of claim 13, wherein determining the user intent includes inputting a prompt to the generative AI model.

15. The storage medium of claim 13, wherein the generative AI model is configured to perform analysis, classification, prediction, or summarization.

16. The storage medium of claim 13, wherein the instructions further cause estimation of the user's emotion using a neural network trained on mapped emotion values.

17. The storage medium of claim 13, wherein retrieving information includes accessing a cloud-based application.

18. The storage medium of claim 13, wherein the instructions further cause secure transmission of data between a terminal device and the server.
